(19) United States
(12) Patent Application Publication
(10) Pub. No.: US 2008/0305174 A1
(43) Pub. Date: Dec. 11, 2008

(54) POLYMERIC NANOCAPSULES FOR USE IN DRUG DELIVERY

(76) Inventors: Robert J. Gyurik, Exeter, NH (US); Zhengmao Li, New Market, NH (US)

Correspondence Address: OSTROLENK FABER GERB & SOFFEN, 1180 AVENUE OF THE AMERICAS, NEW YORK, NY, 100368403, US

(21) Appl. No.: 11/760,083

(22) Filed: Jun. 8, 2007

(51) Int. Cl. A61K 9/51; A61K 38/02; A61K 38/16; A61K 38/28; A61P 3/10; A61K 47/30; A61K 47/32; A61K 47/34

(52) U.S. Cl. 424/497; 514/12; 514/13; 514/14; 514/15; 514/16; 514/17; 514/18; 514/19; 514/2; 514/3; 514/772.1; 514/772.3; 514/772.6

(57) ABSTRACT

The present invention relates to drug delivery formulations that utilize nanocapsules, such as nanovesicles, micelles, lamellae particles, polymersomes, dendrimers, and other nano-size particles of various other fabrications, including those that are known in the art. The invention employs diblock copolymers or single block polymers that hold, adhere to, absorb or encapsulate drug molecules, including, but not limited to, those that heretofore have not been successfully formulated for oral drug delivery, e.g., insulin. Nanocapsule holding, adherence, absorption or encapsulation of such drugs or other molecules enables their delivery via oral or mucosal means.

### BACKGROUND OF THE INVENTION

1. Field of the Invention

Generally speaking, the pertinent field of the present invention relates to drug delivery formulations that form nanoparticles which absorb drugs and deliver them to the body. Such drugs include, for example, peptides and proteins, which are delivered by nanoparticles to the gastrointestinal tract and other portions of the body. More particularly, the technology relating to the general aspect of the invention employs copolymers that form nanocapsules in aqueous solution. These formulations enable the oral and mucosal delivery of polypeptides and macromolecules, e.g., insulin and other polypeptides, that heretofore have not been successfully formulated for oral and mucosal drug delivery.

2. Description of the Related Art

The use of polymers in drug formulations is known. See, for example, U.S. patent publication 2005196343 A1 (Sep. 8, 2005) to Reddy et al. which is said to relate to polymeric nanoparticles, particularly useful in drug and agent delivery; WO 2005079861 A2 (Sep. 1, 2005) to Lee, which is said to relate to a conjugate comprising a chemotherapeutic agent (such as an antitumor drug) conjugated to a water soluble polyamino acid polymer; WO 2005056641 A1 (Jun. 23, 2005) to Kataoka et al. which is said to disclose a coordination complex of a block copolymer comprising a poly(ethylene glycol) segment and a poly(carboxylic acid) segment with diaminocyclohexaneplatinum(II); WO 2005051416 A1 (May 27, 2005) to Pouliquen et al. which is said to disclose pharmaceutical formulations containing stable aqueous colloidal suspensions for the prolonged release of an active ingredient, particularly a protein; KR 2003018549 A (Mar. 6, 2003) to Byun et al. which is said to disclose a phospholipid liposome containing the combination of a negatively charged polymer and a phospholipid; U.S. patent publication 2004136961 A1 (Jul. 15, 2004) to Prokop et al. which is said to disclose compounds comprising a water-based core solution and a water-based corona solution surrounding the core solution; U.S. patent publication 2004052865 A1 (Mar. 18, 2004) to Gower et al. which is said to pertain to core shell particles having a core encapsulated within a calcium carbonate shell with an intermediate layer composed of an amphiphilic compound; WO 2003101476 A1 (Dec. 11, 2003) to Piccariello et al. which is said to relate to active agent delivery systems and specifically compounds that comprise amino acids covalently attached to active agents; JP 2003327693 A2 (Nov. 19, 2003) to Akashi et al. which is said to disclose poly(γ-glutamic acid)(I) derivatives useful for drug carriers; U.S. patent publication 2003194438 A1 (Oct. 16, 2003) to Prescott et al. which is said to disclose an extended-release analgesic for controlling pain comprised of an opioid or non-opioid analgesic drug ionically bound to hyaluronic acid, polyglutamic acid or other ionic polymers; WO 2003079972 A2 (Oct. 2, 2003) to Piccariello et al. which is said to relate to active agent delivery systems, specifically to compounds that comprise amino acids, as single amino acids or peptides, covalently attached to active agents; WO 2003055935 A1 (Jul. 10, 2003) to Li et al. which is said to concern a design for dendritic poly(amino acid) polymer carriers having multiple functional groups at the polymer surface and heterofunctional groups on the poly(amino acid) side chains for drug or diagnostic agent attachment; U.S. patent publication 2003054977 A1 (Mar. 20, 2003) to Kumar et al. which is said to disclose a specified process for preparing a conjugate of poly(glutamic acid) and a therapeutic agent; WO 2003011226 A2 (Feb. 13, 2003) to Ignatious which is said to disclose conjugates of a polymer and biomimetic antagonist to a receptor upregulated at a disease site; WO 2002087497 A2 (Nov. 7, 2002) to Li et al. which is said to disclose conjugate molecules comprising a ligand or a targeting moiety bonded to a polymer spacer, a polymer carrier bonded to the polymer spacer, and a therapeutic agent bound to the polymer carrier (with or without a linker); U.S. patent publication 2002128177 A1 (Sep. 12, 2002) to Latham, which is said to disclose a method of protecting a chemical compound from degradation comprising combining the chemical compound with an amino acid polymer; U.S. patent publication 2002099013 A1 (Jul. 25, 2002) to Piccariello et al., which is said to claim compounds comprising a polypeptide and an active agent covalently attached to the polypeptide and a method for delivery of an active agent to a patient by administering the compound to a patient; WO 2002026241 A1 (Apr. 4, 2002) to Kataoka et al., which is said to disclose a complex comprising cisplatin and a poly(ethylene glycol)/poly(glutamic acid) block copolymer, wherein the cisplatin is enclosed in the copolymer through ligand displacement in which a carboxyl anion of the copolymer is replaced with a chlorine ion of the cisplatin; WO 2001089477 A2 (Nov. 29, 2001) to Mcginniss et al., which is said to disclose compounds and methods for controllably releasing a material or active ingredient from a polymer matrix; WO 2001047501 A1 (Jul. 5, 2001) to Prokop in which it is said that microparticles and nanoparticles prepared from oppositely charged polymers are provided in which a drug is incorporated into the core and is conjugated to one polymer by a Schiff-base crosslink; WO 9918934 A1 (Apr. 22, 1999) to Prokop, which is said to disclose a method of making particles useful in drug delivery, comprising the steps of: contacting polyanionic polymers with cations in a stirred reactor so that polyanions and the cations react to form particles; WO 9851284 A1 (Nov. 19, 1998) to Unger, which is said to be directed to targeted therapeutic delivery systems comprising a gas or gaseous precursor filled microsphere wherein said gas or gaseous precursor filled microsphere comprises an oil, a surfactant, and a therapeutic compound, and WO 9851282 A1 (Nov. 19, 1998) to Unger, which is said to disclose a solid porous matrix formed from a surfactant, a solvent, and a bioactive agent.

In the field of drug delivery, attempts have been made to create systems and materials that successfully deliver insulin to the body by the oral route with a reduced degradation of the insulin by gastrointestinal enzymes. A report by Ghilsai, Drug Delivery Systems, BUSINESS BRIEFING: PHARMAGENERICS (2003), describes a number of approaches to achieving oral delivery of insulin, but states that in most of the approaches described therein, only a small amount of insulin is absorbed in oral administration. Kidron et al. in an abstract, A Novel Peroral Insulin Formulation: Proof of Concept Study in Non-diabetic Subjects, Diabet. Med. 21, 354-357 (2004) reports the oral administration to subjects of an insulin containing delivery agent comprising (sodium N-[8-(2-hydroxybenzoyl)amino]caprylate) (“SNAC”), and purports to disclose that insulin was absorbed through the gastrointestinal tract in a bioactive form. Gowthamarajan et al., Oral Insulin—Fact or Fiction, RESONANCE, May 2003 reports that the strategy of utilizing insulin loaded with nano/microcapsules has been tested a number of times, but that the uptake of insulin via oral route, despite all the precautions, was less than 0.5%. Morishita et al., in Novel Oral Insulin Delivery Systems Based on Complexation Polymer Hydrogels, J. Controlled Release 110 (2006) 587-594 also states that polymeric carriers, lipid-based carriers such as liposomes and solid lipid nanoparticles show low bioavailability as insulin delivery agents. In a press release in Pharmaceutical News dated Jun. 2, 2004, BioSante Pharmaceuticals, Inc. announced what it said were positive results of a preclinical study of a calcium phosphate nanoparticle (CAP) delivery system or oral delivery of insulin. Barclay, Md., in a Jan. 31, 2003 press release by Medscape Medical News, entitled Oral Insulin Effective in Type 2 Diabetes, reported the use of an oral insulin formulation, oral hexyl-insulin monoconjugate 2. Hagan et al. in an abstract entitled Polylactide-Poly(ethylene glycol) Copolymers as Drug Delivery Systems. 1. Characterization of Water Dispersible Micelle-Forming Systems, Langmuir, 12 (9), 2153-2161 (1996) discusses copolymers of polylactide and PEG which are said to self disperse in water to form spherical nonionic micelles as a drug delivery system. Finally, U.S. Pat. No. 7,153,520 to Seo et al. purports to disclose an implant for injection into the body which is associated with a composition for sustained delivery of a drug comprising an amphiphilic diblock copolymer; a poorly water-soluble drug; a biodegradable polymer; and liquid poly(ethylene glycol) (PEG).

Each of the foregoing patents, publications, applications and references is incorporated herein by reference as if fully set forth herein.

### SUMMARY OF THE INVENTION

In general, the present application relates to “nanocapsules,” which term refers to a number of nanoparticles, including, but not limited to, nanovesicles, micelles, lamellae shaped particles, polymersomes, dendrimers, and nano-size particles of various other small fabrications that are known to those in the art. The nanocapsules falling within the general scope of the present invention are drug delivery vehicles that deliver drugs, particularly, peptides and proteins such as insulin, to the gastrointestinal portions of the body where they are absorbed without appreciable degradation by resident enzymes. More particularly, in one aspect of the invention, the nanocapsules are comprised of amphiphilic diblock amino acid or amino acid derivative polymers. In a general aspect of the invention, when these diblocks are in solution, such as the environment of the stomach and intestines, the nanocapsules form and adhere to, or partially adhere to, absorb or encapsulate, the drug molecules of interest, including those that heretofore have not been successfully formulated for oral drug delivery, such as polypeptides and macromolecules, e.g., insulin. Nanocapsule full or partial absorption or encapsulation of such drug molecules enables their delivery via oral or mucosal means including by the inclusion of the diblock formulation in tablet, capsule, caplet, powder, liquid, suspension and other pharmaceutical forms known to those of skill in the art of the drug and pharmaceutical industries.

While other copolymers fall within scope of the present invention, one general aspect of the present application is directed to diblock copolymers, and certain single polymers. For example, conventional thinking is that a triblock polymer is important to form a proper wall for a nanoparticulate drug carrier. However, the applicants herein have discovered that a diblock polymer form is a particularly effective nanoparticulate drug carrier.

Thus, in general, the present invention pertains to molecules with a particular mechanism of action. The linear amphiphilic molecules herein described comprise a diblock polymer (“A-B Polymer”) with the A chain being at least partially hydrophobic and the B chain being at least partially hydrophilic. Further, in one aspect of the present invention, the A and B copolymer units comprise amino acids or derivatized amino acids.

In summary fashion, the diblock polymers comprising one aspect of the present invention can be described as follows:

A diblock polymer comprising polymer blocks A and B, wherein
A is at least a partially hydrophobic block and
B is at least a partially hydrophilic block, and

wherein A and B further comprise amino acids or derivatized amino acids.

When put into a hydrophilic environment e.g., an aqueous solution, the polymer will spontaneously form nanocapsules, e.g, micelles and/or lamellae shaped particles, with the lipophilic end facing inward to ‘hide’ from the water. The nanocapsule assembly can resemble a particle, since the individual lipophilic ends are attracted with a force called Van der Waals attraction. Additionally, the process of nanocapsule (e.g., micelle) formation is sometimes called hydrophobic bonding.

Thus, one object of the present invention is to provide molecules that are designed to deliver drugs by routes difficult to pursue by other means, such as oral delivery of sensitive drug molecules such as insulin and other peptides or polypeptides.

Another object of the invention is to provide nanocapsules that at least partially adhere to, absorb and/or encapsulate drug molecules in the body.

Another object of the invention is to provide a delivery vehicle for drugs to be absorbed into the stomach, intestines and/or gastrointestinal tract without degradation of such drugs, e.g., insulin, by enzymes resident in those areas of the body.

Another object of the invention is to provide a new oral or mucosal delivery means for drug molecules in general.

Another object of the invention of the present invention is to provide a delivery system, such as a tablet, capsule, liquid, suspension, intravenous, intraperitoneal, subcutaneous, intrathecal, and opththalmic means for the delivery or administration to a patient of insulin and other polypeptides and proteins.

Another object of the invention is to provide a diblock polymer that forms nanocapsules when the polymer is introduced into aqueous media.

Another object of the invention is to provide nanocapsules that are capable of at least partially absorbing, encapsulating or adhering to drug molecules and serving as drug carriers.

Additional advantages, uses and features of the aspects of the invention described herein will be apparent to those of skill in the art from the detailed description which follows, including the accompanying example and drawings.

### BRIEF DESCRIPTION OF THE DRAWINGS

FIG. 1 is a graphical representation of the results of a study measuring inter alia the concentration over time of glucose and insulin in an insulin formulation of the present invention wherein said insulin formulation was administered orally to an animal.

FIG. 2 is a graphical representation of the results of a study measuring inter alia concentration over time of insulin and glucose levels wherein insulin was not formulated in accordance with the present invention and wherein the insulin was administered orally to an animal.

### DETAILED DESCRIPTION OF THE INVENTION

Numerous technologies and entities are commonly embraced by the catch all terms “nanotechnology” and “nanoparticles.” A “nanoparticle” is a term that relates to a number of entities, many of which are known to one of skill in the art and which are incorporated herein by reference. One thing in common, however, is that nanoparticles or “nanostructures” are usually sufficiently small to be measured in nanometers.

A term used in this application, “nanocapsule” refers to a number of nanoparticles, including, but not limited to, nanovesicles, micelles, lamellae shaped particles, polymersomes, dendrimers, and other nano-size particles of various other small fabrications that are known to those in the art. The definitions and understandings of the entities falling within the scope of nanocapsule are known to those of skill in the art, and such definitions are incorporated herein by reference and for the purposes of understanding the general nature of the subject matter of the present application. However, the following discussion is useful as a further understanding of some of these terms.

For example, a “micelle”, a useful article in the employment of a general aspect of the present invention, can generally be thought of as a small—on the order of usually nanometers in diameter—aggregate of amphiphilic linear molecules having a polar, or hydrophilic end and an opposite non-polar, or hydrophobic end. These linear molecules can be comprised of simple molecules, or polymeric chains. A micelle can also be referred to as an aggregate of surfactant molecules dispersed in a liquid colloid. A typical micelle in aqueous solution can form an aggregate with the hydrophilic “head” regions in contact with surrounding solvent, and the sequestering of the hydrophobic tail regions in the micelle center. Other and similar definitions, descriptions and understandings of micelles are also known to those of skill in the art and are incorporated herein by reference.

“Polymersomes” can, in general, be thought of as bilayered membranes of amphiphilic synthetic polymers, which are similar in some respects to liposomes, which use naturally occurring lipids. While having some of the properties of natural liposomes, polymersomes exhibit increased stability and reduced permeability. Other and similar definitions, descriptions and understandings and of polymersomes are also known to those of skill in the art and are incorporated herein by reference.

“Dendrimers” have descriptions, definitions and understandings in the literature. For example, and without limitation and including other and similar definitions, descriptions and understandings in the art, the term dendrimer from the Greek word, “dendron”, for tree, can refer to a synthetic, three-dimensional molecule with branching parts. Descriptions and understandings of dendrimers can be gleaned from Holister et al., DENDRIMERS, Technology White Papers nr. 6, pub. October 2003 by cientifica, as well as the other literature published by those skilled in the art on dendrimers, all of which are incorporated herein by reference.

“Lamella” is a term whose definitions, descriptions and understandings are also known to those of skill in the art and which are incorporated herein by reference. In a very general sense, lamella or lamellae refers to plate-like, gill-shaped or other layered structures.

The definitions, descriptions and understandings of “nano-vesicle” are well known to those of skill in the art, and are incorporated herein by reference. For example, “nanovesicle” can refer to a variety of small sac, sac-like or globular structures capable of containing fluid or other material therein.

Generally speaking, the present invention relates to drug delivery formulations that utilize nanocapsules to deliver drugs, particularly, peptides and proteins such as insulin, to the gastrointestinal portions of the body where they are absorbed without appreciable degradation by resident enzymes. The nanocapsules are generally comprised of block and diblock polymers and are formed in solution or suspension where they can be combined with the drug molecule of interest. In a general aspect of the invention, the diblock polymers are comprised of amphiphilic amino acid, or amino acid derivative, copolymers. When these diblocks are in solution or suspension, nanocapsules form and can adhere to, or partially adhere to, and can at least partially absorb or encapsulate, drug molecules, including, but not limited to, those that heretofore have not been successfully formulated for oral drug delivery, such as polypeptides and macromolecules, e.g., insulin, insulin derivatives and analogues, growth hormones and analogues thereof, eyrthropoeitins, anti-inflammatory peptides, anti-aging peptides, atrial natriurectic peptides, brain injury derived peptides, Calcitonin, defensins, deltorphins, dermorphins and analogues thereof, BAM peptides, α-casein exorphins, dynorphins, endomorphins, endorphins, enkephalins, gluten exorphins, kyotorphins, methorphamide, neoendorphins, syndyphalins, valorphin, dynorphin and analogues and sequences thereof, enterostatins, Ghrelins, glucagons and glucagon-like peptides such as GLP-1 and GLP-2, gonadotropin releasing hormones, growth hormone releasing hormones, insulino-tropic compounds, kyotorphins, leptin and fragments thereof, secretins, thymosins and fragments thereof, transforming growth factors and fragments thereof, tuftsin, tumor necrosis factors and related peptides, and VIP, Prepro VIP, and analogs and fragments thereof. A nanocapsule falling within the scope of the present invention effects full or partial adherence, absorption or encapsulation of such drug molecules and thereby enables their delivery via oral or mucosal means. One aspect of the present invention also employs a single polymer such as is exemplified in some of the alternative embodiments described below.

Thus, one aspect of the present invention is a type of copolymer called a “block copolymer”, a term whose definitions and understandings are well known in the art and which are incorporated by reference herein. In a general sense, block copolymers are comprised of two or more polymer subunits linked by covalent bonds. Block copolymers with two or three distinct blocks are called diblock copolymers and triblock copolymers, respectively. Copolymers may also be described in terms of the existence of or arrangement of branches in the polymer structure. Linear copolymers consist of a single main chain whereas branched copolymers consist of a single main chain with one or more polymeric side chains. Block copolymers are made up of blocks of different polymerized monomers. Triblocks, tetrablocks, multiblocks can be made. Block copolymers are of interest because they can “microphase separate” to form periodic nanostructures.

While other copolymers fall within scope of the invention, one aspect of the present application is directed to diblock copolymers, although single chain and other polymers may be used. As mentioned above, conventional thinking is that a triblock polymer is important to form a proper wall for a nanoparticulate drug carrier. However, the applicants herein have found that the diblock polymer form, and in some cases the single polymer form, is a particularly advantageous and effective nanoparticulate drug carrier. According to one general aspect of the present invention, the evidence strongly suggests that the diblock polymers of the present invention form nanocapsules such as those with a lamella-like configuration, alone or together with nanocapsules of micelle configuration. The lipid block of the nanocapsule (e.g., n-butyl-poly-l-lactide) intercalates or interweaves in aqueous solution and the polar group of the nanocapsule (e.g., polyglutamic acid with associated drug, e.g., insulin) faces the aqueous solution both inside and out when the nanocapsules are formed. The nanocapsule assembly resembles a particle, since the individual lipophilic ends are attracted with a force called Van der Waals attraction. The process of nanocapsule formation is sometimes called hydrophobic bonding. In accordance with the aforementioned mechanism of action, when the nanocapsule/drug is in the physiological media of the gastrointestinal tract, the drug that has been formulated with it is protected by the nanocapsule so it may be absorbed intact by the tissues of the body and introduced into the blood stream before it can be degraded by gastrointestinal enzymes. Experimental support for this mechanism is set forth in the example below wherein effective oral delivery of insulin utilizing this aspect of the invention is demonstrated.

Hagan and Seo et al., referred to above, concerns, inter alia, PEG-PLA diblock polymers. Although they are diblock polymers, they differ from the poly(lactic acid)-poly(glutamic acid) diblock aspect of the present invention which uses poly(glutamic acid) as the hydrophilic block, instead of polyethylene glycol, PEG. PEG-PLA diblock polymers can only deliver hydrophobic drugs, if at all. One aspect of the present invention is that it can deliver hydrophilic polypeptides and proteins, e.g. insulin. Further, PEG has no charges on the polymer backbone, while poly(glutamic acid), as used within the scope of the present invention, has negative charges at neutral and basic pH, and thus can absorb or encapsulate hydrophilic proteins for drug delivery.

Also, both blocks of the diblock polymers included within the present invention are biodegradable. In addition, the diblock polymer nanocapsule structures encapsulate aqueous solutions with hydrophilic proteins inside the nanocapsule. As mentioned above, the poly(glutamic acid) on the outer surface of the nanocapsule adsorbs hydrophilic proteins. The PEG-PLA block polymers of the prior art are not known to form nanocapsules. Rather they form a hydrophobic PLA hard core with PEG sticking out into the aqueous phase of a solution. Further, PEG does not absorb proteins, and thus a PEG-PLA drug delivery agent mainly depends on the encapsulation of hydrophobic drugs in the PLA core, not the PEG.

In general, the present invention pertains to amphiphilic molecules comprising a diblock (“A-B Polymer”) with the A chain being at least partially hydrophobic and the B chain being at least partially hydrophilic. Further, in one aspect of the present invention, the A and B copolymers comprise amino acids or derivatized amino acids. Still further, in another aspect of the invention, the B polymer alone is sufficient to provide the nanocapsule drug delivery agent. In summary fashion, the diblock polymers comprising one aspect of the present invention can be summarized as follows:

A diblock polymer comprising polymer blocks A and B, wherein
A is at least a partially hydrophobic block and
B is at least a partially hydrophilic block, and

wherein A and B further comprise amino acids or derivatized amino acids.

The nature of the amphiphilic diblock amino acid (or derivatized amino acid) polymer of the present invention is such that when it is in suspension or solution it will spontaneously form nanocapsules, with a lipophilic end facing inward to ‘hide’ from water, which at least partially, encapsulate, absorb, partially or fully, or adhere to, the drug molecule of interest added to the nanocapsules. Thus, as aforesaid, when the nanocapsules are in the aqueous media of the stomach, intestines and gastrointestinal tract, they are absorbed by the tissues and the drug, e.g., a polypeptide or protein, is delivered safely and without appreciable degradation by the body's enzymes.

The type of molecules of the present invention are generally designed and are useful to deliver drugs by routes difficult to pursue by other means, such as oral delivery of sensitive drug molecules such as insulin and other peptides or polypeptides. However, other small conventional drug molecules are included within the present invention, and the success depends upon the ionic nature of the compounds, and whether they will form complexes with the diblocks. Since the nanocapsules formed by the copolymers of this invention adhere to, absorb and/or encapsulate the drug molecule, the drug may be delivered orally or through mucosal membranes. Further, since the nanocapsules formed as described herein are capable of absorbing, encapsulating or adhering to a drug, they are highly useful as drug carriers. Because of their size and sensitive drug carrier capabilities, the nanocapsules of the present invention provide unique and precise drug carrier capabilities. Thus, one advantage of the present invention is that polypeptide and protein drugs, e.g., insulin, and other macromolecules may be delivered orally, or through other mucosal membranes, whereas other technologies have failed or had severe shortcomings. In addition, other delivery routes falling within the scope of the present invention include intravenous, intraperitoneal, subcutaneous, intrathecal, opththalmic, intranasal, liquid, inhaler and other delivery routes known in the art.

A description of one embodiment of the A-B block copolymer of the present invention is as follows:

| Exemplary | Amount | |
|---|---|---|
| Composition | (% w/w) | General Composition |
| Block A: | 50 | Block A is a lipophilic polymer |
| n-butyl-poly-1-lactide | | approximately 100 units or as |
| | | determined suitable for delivery of |
| | | insulin or other peptides by the oral |
| | | route. |
| Block B: | 50 | Block B is a hydrophilic amino acid |
| poly-1-glutamic acid | | polymer, approximately 100 units |
| | | or as determined suitable for |
| | | delivery of insulin or other peptide |

These A-B polymer compositions are easily made using standard wet chemical methods. The major step, the coupling of A and B, is usually accomplished under anhydrous conditions using standard peptide dehydrative coupling reactions, such as is achieved with dicyclohexylcarbodiimide. Other coupling reagents are known to practitioners skilled in the art as being useful for coupling, and include polymer fusion reagents and peptide coupling reagents that can carry out the joining of the two polymeric blocks. Diblock copolymers can also be made using living polymerization techniques, such as atom transfer free radical polymerization (ATRP), reversible addition fragmentation chain transfer (RAFT), ring-opening metathesis polymerization (ROMP), and living cationic or living anionic polymerizations.

### EXAMPLE

### Synthesis of Diblock Copolymer

The amphiphilic diblock copolymer, poly(lactic acid)-poly(glutamic acid), was made by living polymerization of individual poly(lactic acid) and poly(glutamic acid) blocks, then coupling the two blocks together. In the diblock polymer used in the following pig study, each poly(lactic acid) block has approximately 150 lactic acid repeating units, and each poly(glutamic acid) block has approximately 100 glutamic acid repeating units, which were confirmed by Gel Permeation Chromatography (“GPC”) and Multi-Angle Laser Light Scattering (“MALLS”).

After the coupling reaction, the diblock polymer was precipitated and washed in water. 30 mL of aqueous polymer suspension with about 3% polymer content was sonicated for 15 minutes, then 10 mL of 1% insulin solution was added. The final pH was 7.4. In the final formulation, there were 2.2% diblock nanocapsule and 0.25% insulin at pH 7.4 (“Formulation A”). The insulin was added after the diblock nanocapsules were formed, so insulin was adsorbed on the outside of the nanocapsules, or freely floating in the aqueous phase.

### Yucatan Minipig Study

Yucatan pigs were prepared for the study with the surgical implantation of a jugular catheter for easy blood collection. Baseline venous blood specimens were collected just prior to the dosing treatment and blood was thereafter sampled at 0 (just before treatment), 30, 60, 90, 120, 150 and 240 minutes after treatment. Each pig was monitored with a hand-held commercial glucometer (Lifescan, J&J; One Touch Fast Take™) at each blood collection time to ensure animal wellness, and to give an immediate indication of any biological activity as is verified by glucose reduction compared to the baseline levels.

The blood was collected into sodium heparinized plastic tubes. The plasma was retrieved and stored at −20° C. until analyzed for insulin and glucose.

Heparinized plasma was analyzed for insulin concentration using a commercial ELISA assay for insulin (Linco Research, Inc.; Human Insulin Specific ELISA Kit, Cat# EZHI-14K). Insulin was reported in micro International Units/milliliter of plasma (μU/mL).

The pigs were dosed with either insulin solution at 0.25% (Control) or nanocapsule associated insulin at 0.25% (Formulation A). Each formulation was dosed by oral gavage, 4 mL of formulation, followed by 4 ml of saline wash to rinse the tubing.

### Results and Discussion

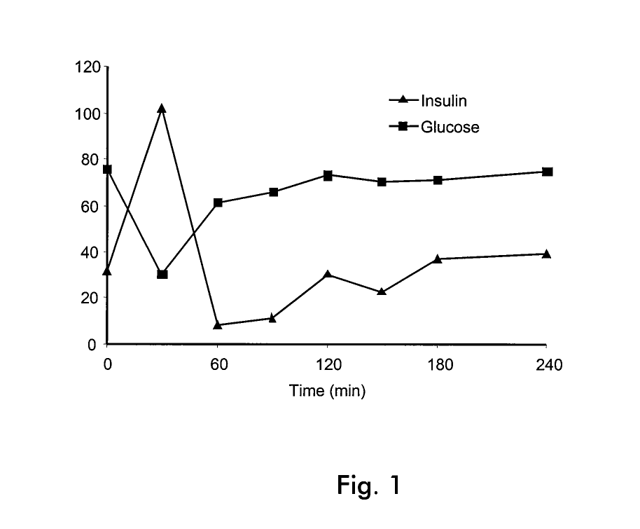

After administering 4 mL of diblock (Formulation A) orally, the pigs showed glucose reduction at about 30 minutes. See Table 1 and FIG. 1. This pharmacodynamic result was confirmed by the pharmacokinetic blood insulin concentration increase. The insulin assay measured both the endogenous pig insulin and human insulin. Table 1 and FIG. 1 show the performance of oral 4 mL 0.25% insulin (Formulation A) with 2.2% poly(lactic acid)-poly (glutamic acid) nanocapsules at pH 7.4. Glucose levels were also measured and obtained. A graphical representation of these data is shown in FIG. 1.

TABLE 1
D12P1 (O, 4 mL F)

| Time | Insulin | Glucose |
|---|---|---|
| (min) | (uU/mL) | (mg/dL) |
| 0 | 31.315 | 76 |
| 30 | 102.18 | 30 |
| 60 | 8.2559 | 61 |
| 90 | 10.86 | 66 |
| 120 | 30.544 | 73 |
| 150 | 22.803 | 70 |
| 180 | 37.159 | 71 |
| 240 | 39.395 | 75 |

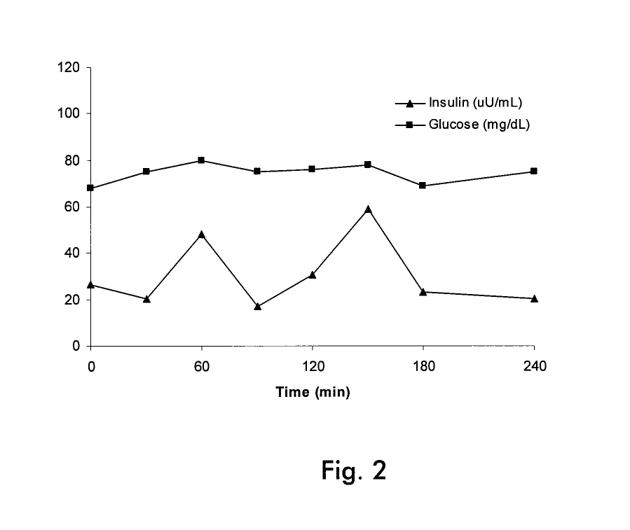

The result of administration of 4 mL of 0.25% insulin solution (without nanocapsules) orally to the pigs as a control is illustrated in Table 2 and FIG. 2. The results of the control experiment are in accord with the well known fact that insulin without protection can not survive the stomach and intestine, which is the reason why, to the inventors knowledge, insulin has not yet been taken orally in an effective manner. The insulin and glucose results for this control (see Table 2 and FIG. 2) confirmed that the pig glucose was unchanged; it also showed the baseline insulin level in the pigs. FIG. 2 also graphically shows the performance of oral 4 mL 0.25% insulin solution at pH 7.4.

In sum, Table 2 shows the experimental results of oral 4 mL 0.25% insulin solution at pH 7.4. Glucose levels were also measured and obtained. A graphical representation of these data are shown in FIG. 2.

TABLE 2
D10P2 (O, 4 mL A)

| Time | Insulin | Glucose |
|---|---|---|
| (min) | (uU/mL) | (mg/dL) |
| 0 | 26.304 | 68 |
| 30 | 20.214 | 75 |
| 60 | 48.063 | 80 |
| 90 | 17.158 | 75 |
| 120 | 30.544 | 76 |
| 150 | 59.066 | 78 |
| 180 | 23.256 | 69 |
| 240 | 20.214 | 75 |

### CONCLUSION

The experimental results confirm that a poly(lactic acid)-poly(glutamic acid) nanocapsule can protect insulin at least partially absorbed on or in the nanocapsule upon transit through the stomach and the intestine, and deliver insulin to the blood stream, even without the use of enteric coating on the formulation. The interaction between the insulin molecule and the poly(glutamic acid) on the outer surface of the nanocapsule is one key factor. The poly(glutamic acid) acts as a coating protecting the insulin. The inventors have deduced that in the acidic environment in the stomach, poly(glutamic acid) is protonated, becomes hydrophobic and protects the adsorbed insulin inside. In the intestine, protonated poly(glutamic acid) lost protons due to the higher pH and stretched out due to charge repulsion, then released the insulin. The poly(lactic acid) block functioned to aggregate the molecules into nanocapsules of uniformly small particle size that enabled them to be uptaken by an as yet unknown mechanism through the intestinal mucosal cells.

As the results of these experiments show, the present invention is a breakthrough discovery in the field of oral insulin delivery and is a new way of delivering insulin orally. As shown by the data presented above and the graphical representations in FIGS. 1 and 2, the polymeric nanocapsule formulations of the present invention show superior results in the drug delivery of insulin.

The following are further examples of selected embodiments of the invention in addition to that described above. They are not meant to be limiting of the scope of the invention and other embodiments will be apparent to those skilled in the art.

A second embodiment of the invention comprises a composition comprising a diblock polymer having components A and B, wherein A can be at least a partially hydrophobic block, B can be at least a partially hydrophilic block, and wherein said composition may form one or more of the following entities, for example, in solution or suspension: nanovesicles, micelles, lamellae particles, polymersomes, dendrimers, and other nano-size particles of various fabrications, including, but not limited to, those that are known to those of skill in the art.

A third embodiment of the invention comprises the composition of the second embodiment wherein a drug may be at least partially absorbed or encapsulated by, or adhered to, one or more of the following entities: nanovesicles, micelles, lamellae particles, polymersomes, dendrimers, and other nano-size particles of various fabrications, including, but not limited to, those that are known to those of skill in the art.

A fourth embodiment of the invention comprises the composition of the second embodiment wherein a drug may be at least partially absorbed or encapsulated by, or adhered to, nanocapsules.

A fifth embodiment of the invention comprises the composition of the second embodiment wherein the A block may be, for example, a polylactide, polycaprolactide, or polyglycolide composition, of either enantiomeric, racemic, or other isomeric forms such as meso.

A sixth embodiment of the invention comprises the composition of the second embodiment wherein the B block may be, for example, polyamino acids with ionic nature, of either enantiomeric, racemic, or other isomeric forms such as meso.

A seventh embodiment of the invention comprises the composition of the second embodiment wherein the B block may be polyaminoacids with anionic nature, for example, polyglutamic and polyaspartic amino acids, or copolymers of the two.

An eighth embodiment of the invention comprises the composition of the second embodiment wherein the B block may be polyaminoacids with cationic nature, for example, polylysine, polyarginine, polyhistidine, or copolymers of the three taken two or three at a time.

A ninth embodiment of the invention comprises the composition of the third embodiment of the invention wherein the drug is a polypeptide or macromolecule.

A tenth embodiment of the invention comprises the composition of the third embodiment of the invention wherein the drug is a polypeptide or macromolecule.

An eleventh embodiment of the invention comprises the composition of the third embodiment of the invention wherein the drug may be insulin or derivative or analog of insulin.

A twelfth embodiment of the invention comprises the composition of the third embodiment of the invention which differs, however, in that instead of having A and B block copolymers, the composition may comprise only the B block as a polyamino acid capable of complexing with the drug in such a way as to protect the drug before being uptaken by the intestine after oral delivery.

A thirteenth embodiment of the invention comprises the composition of the sixth embodiment of the invention which differs, however, in that instead of having A and B block copolymers, the composition may comprise only the B block as disclosed in embodiments 6 through 8.

A fourteenth embodiment of the invention comprises the composition of the seventh embodiment of the invention which differs, however, in that instead of having A and B block copolymers, the composition may comprise only the B block as disclosed in embodiments 6 through 8.

A fifteenth embodiment of the invention comprises the composition of the eighth embodiment of the invention which differs, however, in that instead of having A and B block copolymers, the composition may comprise only the B block as disclosed in embodiments 6 through 8.

Further embodiments of the invention comprise the composition of the second through fifteenth embodiments of the invention wherein one or more of the blocks may vary in length between approximately 10 and 500 monomeric units, preferably between approximately 25 and 200 units, and most preferably between approximately 50 and 150 units.

This application and the various aspects of the invention herein are not limited to the embodiments illustrated above, and other embodiments will be apparent to those of skill in the art. The embodiments described above are meant to be illustrative and not limiting.

1. A composition comprising:
a diblock copolymer comprising polymer blocks A and B, wherein
A is at least a partially hydrophobic block and
B is at least a partially hydrophilic block, and
wherein A and B further comprise amino acids or derivatives of amino acids.

2. A composition according to claim 1 wherein block A comprises an n-butyl-poly-l-lactide polymer or a derivative thereof.

3. A composition according to claim 1 wherein block B comprises a poly-l-glutamic acid polymer or a derivative thereof.

4. A composition according to claim 1 wherein the diblock polymer comprises poly(lactic acid)-poly(glutamic acid) or a derivative thereof.

5. A composition according to claim 1 wherein the A block comprises a polylactide, polycaprolactide, or polyglycolide, or derivatives thereof, including such entities in either enantiomeric, racemic or other isomeric forms such as meso.

6. A composition according to claim 1 wherein the B block comprises polyaminoacids, or derivatives thereof, with ionic nature, including polyaminoacids in enantiomeric, racemic, or other isomeric forms such as meso.

7. A composition according to claim 1 wherein the B block comprises polyaminoacids, or derivatives thereof, with anionic nature, including polyaminoacids in enantiomeric, racemic, or other isomeric forms such as meso.

8. A composition according to claim 1 wherein the B block comprises polyaminoacids, or derivatives thereof, with cationic nature.

9. A composition according to claim 1 wherein the B block comprises polyglutamic or polyaspartic amino acids, or derivatives thereof, or copolymers of the two.

10. A composition according to claim 1 wherein the B block comprises polylysine, polyarginine, polyhistidine, or derivatives thereof, or any combinations of copolymers thereof.

11. A composition according to claim 1 wherein said diblock polymer forms nanocapsules.

12. A composition according to claim 11 wherein the nanocapsules comprise nanoparticles.

13. A composition according to claim 11 wherein the nanocapsules comprise micelles.

14. A composition according to claim 11 wherein the nanocapsules are lamella shaped.

15. A composition according to claim 11 wherein the nanocapsules comprise both micelles and lamellae shaped particles.

16. A composition according to claim 11 wherein the nanocapsules comprise polymersomes.

17. A composition according to claim 11 wherein the nanocapsules comprise nanovesicles.

18. A composition according to claim 11 wherein the nanocapsules comprise dendrimers.

19. A composition according to claim 11 wherein a drug is adhered to, or present on or in, all or part of the nanocapsules.

20. A composition according to claim 11 wherein a drug is at least partially absorbed, or encapsulated by, all or part of the nanocapsules.

21. A composition according to claim 19 wherein the drug is a peptide, polypeptide or protein.

22. A composition according to claim 19 wherein the drug is a macromolecule.

23. A composition according to claim 19 wherein the drug is insulin.

24. A composition according to claim 19 wherein the drug is a derivative or analogue of insulin.

25. A composition according to claim 20 wherein the drug is a peptide, polypeptide or protein.

26. A composition according to claim 20 wherein the drug is a macromolecule.

27. A composition according to claim 20 wherein the drug is insulin.

28. A composition according to claim 20 wherein the drug is a derivative or analogue of insulin.

29. A composition according to claim 1 wherein either or both of blocks A and B may vary in length between approximately 10 and 500 monomeric units.

30. A composition according to claim 1 wherein either or both of blocks A and B may vary in length between approximately 25 and 200 monomeric units.

31. A composition according to claim 1 wherein either or both of blocks A and B may vary in length between approximately 50 and 150 units.

32. A composition according to claim 1 except wherein the composition comprises only the B block comprising a polyamino acid, or a derivative thereof, capable of complexing with a drug such that the B block protects the drug before being uptaken by the intestine.

33. A composition according to claim 1 except wherein the composition comprises the B block but not the A block.

34. A composition according to claim 6 except wherein the composition comprises the B block but not the A block.

35. A composition according to claim 7 except wherein the composition comprises the B block but not the A block.

36. A composition according to claim 8 except wherein the composition comprises the B block but not the A block.

37. A composition according to claim 9 except wherein the composition comprises the B block but not the A block.

38. A composition according to claim 10 except wherein the composition comprises the B block but not the A block.

39. A composition according to claim 1 wherein the diblock polymer comprises a poly(lactic acid) block comprising about 150 lactic acid repeating units, and a poly(glutamic acid) block comprising about 100 glutamic acid repeating units.

40. A nanocapsule comprising a composition according to claim 1.

41. A nanocapsule according to claim 40 wherein the nanocapsule comprises a nanoparticle.

42. A nanocapsule according to claim 40 wherein the nanocapsule comprises a micelle.

43. A nanocapsule according to claim 40 wherein the nanocapsule comprises a lamella shaped structure.

44. A nanocapsule according to claim 40 wherein the nanocapsule comprises a polymersome.

45. A nanocapsule according to claim 40 wherein the nanocapsule comprises a nanovesicle.

46. A nanocapsule according to claim 40 wherein the nanocapsule comprises a dendrimer.

47. A method of making an amino acid or amino acid derivative diblock copolymer that forms nanocapsules, comprising:
polymerizing two individual amino acid or amino acid derivative polymeric blocks;
coupling the two blocks together to form a diblock polymer;
precipitating the diblock polymer after the coupling;
washing the precipitated diblock polymer;
forming a diblock polymer suspension;
sonicating the diblock polymer suspension, and
forming nanocapsules in the suspension.

48. A method according to claim 47 wherein a drug is added to the nanocapsules.

49. A method according to claim 48 wherein the drug is a peptide, polypeptide or protein.

50. A method according to claim 48 wherein the drug is insulin.

51. A method according to claim 48 wherein the drug is an insulin derivative or analogue.

52. A method of treating a patient in need of insulin comprising administering to said patient a pharmaceutically acceptable amount of a composition of claim 23.

53. A method of treating a patient in need of insulin comprising administering to said patient a pharmaceutically acceptable amount of a composition of claim 24.

54. A method of treating a patient in need of insulin comprising administering to said patient a pharmaceutically acceptable amount of a composition of claim 27.

55. A method of treating a patient in need of insulin comprising administering to said patient a pharmaceutically acceptable amount of a composition of claim 28.
